(19) United States
(12) Patent Application Publication
(10) Pub. No.: US 2013/0310817 A1
(43) Pub. Date: Nov. 21, 2013

(54) DEVICES AND METHODS FOR TISSUE TREATMENT ACROSS A LARGE SURFACE AREA

(71) Applicant: Solta Medical Inc., Hayward, CA (US)

(72) Inventor: George Frangineas, Freemont, CA (US)

(73) Assignee: SOLTA MEDICAL INC., Hayward, CA (US)

(21) Appl. No.: 13/948,390

(22) Filed: Jul. 23, 2013

Related U.S. Application Data
(63) Continuation of application No. 13/210,893, filed on Aug. 16, 2011.
(60) Provisional application No. 61/407,543, filed on Oct. 28, 2010.

(51) Int. Cl. A61B 18/20; A61N 5/06

(52) U.S. Cl. CPC A61B 18/203; A61N 5/0613
USPC 606/9; 607/89

(57) ABSTRACT

Light sources and methods for spreading a beam of electromagnetic radiation. The light sources include a scattering element with an outlet and an angular-selective element with an inlet spatially disposed between the outlet of the scattering element and an electromagnetic radiation source. The beam enters the inlet traveling in a direction of propagation and propagates through the beam spreader to the outlet for transmission from the outlet. The scattering element includes a scattering medium configured to scatter the electromagnetic radiation in the beam to provide a two-dimensional spatial distribution for intensity that is substantially uniformly across the outlet. The angular-selective element is configured to reflect a majority of the electromagnetic radiation of the first beam scattered by the scattering medium in a direction opposite to the propagation direction and reaching the angular-selective element.

### CROSS-REFERENCE TO RELATED APPLICATIONS

This application is a continuation of application Ser. No. 13/210,893, filed Aug. 16, 2011, which claims the benefit of U.S. Provisional Application No. 61/407,543, filed Oct. 28, 2010, each of which is hereby incorporated by reference herein in its entirety for all purposes.

### BACKGROUND

The invention relates generally to devices and methods for treating tissue with electromagnetic radiation and, in particular, to light sources and methods that uniformly irradiate the skin surface over a large surface area for tissue and/or skin surface treatment with electromagnetic radiation.

Electromagnetic radiation (EMR) has found use in a wide variety of cosmetic and medical applications, including uses in dermatology. For most dermatological applications, the EMR treatment is performed with a device that delivers the EMR to the tissue surface. Conventional EMR treatments are typically designed to deliver radiation to induce a particular chemical reaction within the targeted tissue, to deliver radiation to cause an increase in tissue temperature, to deliver radiation to damage the targeted tissue, to cause a change at the skin surface, or to modify matter on the skin surface.

Improved light sources and methods are needed for treating tissue with coherent electromagnetic radiation characterized by a relatively uniform irradiance over a large surface area.

### SUMMARY

In another embodiment of the invention, a light source is provided for irradiating a tissue surface. The light source includes a first electromagnetic radiation (EMR) source configured to generate a first beam of electromagnetic radiation and an angular-selective element positioned between the first EMR source and the tissue surface. The angular-selective element is arranged relative to the first EMR source such that the first beam of electromagnetic radiation propagates in a propagation direction through the angular-selective element to reach the tissue surface. The angular-selective element is configured to reflect a majority of the electromagnetic radiation of the first beam scattered by the tissue in a direction opposite to the propagation direction and reaching the angular-selective element. Scattering by the angular-selective element, in combination with the tissue as a scattering element, reduces or eliminates the spatial coherence of the incident beam.

In another embodiment of the invention, a light source is provided that includes an electromagnetic radiation (EMR) source configured to generate a beam of electromagnetic radiation and a beam spreader. The beam spreader includes a scattering element with an outlet and an angular-selective element with an inlet spatially disposed between the outlet of the scattering element and the first EMR source such that the first beam enters the inlet traveling in a direction of propagation and propagates through the beam spreader to the outlet for transmission from the outlet. The scattering element is comprised of a scattering medium configured to scatter the electromagnetic radiation in the first beam to provide a two-dimensional spatial distribution for intensity that is substantially uniformly across the outlet. The angular-selective element is configured to reflect a majority of the electromagnetic radiation of the first beam scattered by the scattering medium in a direction opposite to the propagation direction and reaching the angular-selective element. Scattering by the angular-selective element, in combination with the scattering medium of the scattering element, reduces or eliminates the spatial coherence of the incident beam.

In a specific embodiment, the electromagnetic radiation is projected from the outlet onto a surface area of skin and a dermatological treatment is performed.

In an embodiment of the invention, a method is provided for spreading a beam of electromagnetic radiation. The method includes transmitting the electromagnetic radiation of the beam in a propagation direction through an angular-selective element and into a scattering element. The electromagnetic radiation is scattered in a scattering medium of the scattering element such that an area of a two-dimensional spatial distribution of intensity output at an outlet of the scattering element is substantially uniformly across the outlet. The method further includes reflecting a majority of the electromagnetic radiation that is scattered by the scattering medium in a direction opposite to the propagation direction and that reaches the angular-selective element.

In a specific embodiment, the electromagnetic radiation is employed to perform a dermatological treatment.

### BRIEF DESCRIPTION OF THE SEVERAL VIEWS OF THE DRAWINGS

The accompanying drawings, which are incorporated in and constitute a part of this specification, illustrate various embodiments of the invention and, together with a general description of the invention given above and the detailed description of the embodiments given below, serve to explain the embodiments of the invention.

FIG. 1 is a diagrammatic view of a light source in accordance with an embodiment of the invention.

FIG. 1A is a bottom view of the light source of FIG. 1.

FIG. 2 is a diagrammatic view of a light source in accordance with an alternative embodiment of the invention.

FIG. 3 is a diagrammatic view of a light source in accordance with an alternative embodiment of the invention.

FIG. 4 is a diagrammatic view of a light source in accordance with an alternative embodiment of the invention.

### DETAILED DESCRIPTION

Generally, the embodiments of the light source described herein are configured to couple power from a laser beam to skin, or other tissue, with a uniform or substantially uniform illumination across a surface area of the skin. The light source spreads the spot size of the laser beam such that the cross-sectional area of the coherent light discharged from an exit side of the light source that is larger than the cross-sectional area of the laser beam on an entrance side of the device. The light source is a device that operates based upon the principles of multiple scattering of the laser beam as the laser beam is transferred from the entrance side to the exit side of the device. The uniform or substantially uniform illumination lacks spots of divergently high intensity and is achieved with low loss of beam intensity. The beam spreader of the light source scrambles the spatial coherence of a laser beam so that the laser beam is not focused or concentrated upon exit from the light source, but does so with high photon efficiency.

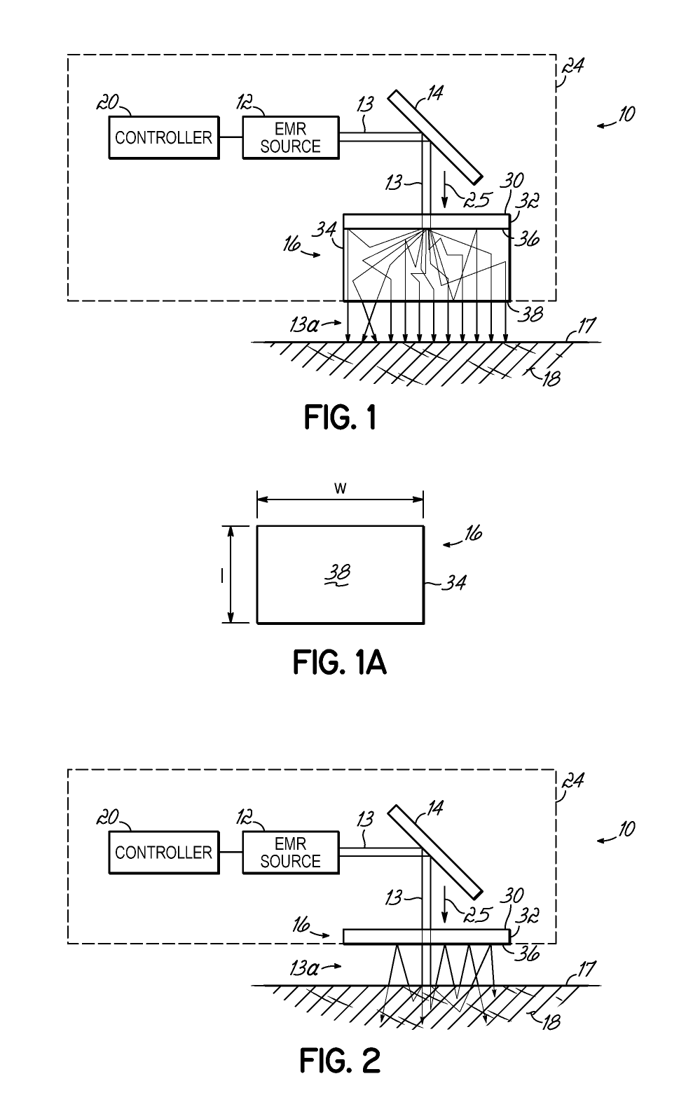

With reference to FIGS. 1, 1A and in accordance with an embodiment of the invention, a light source 10 includes an electromagnetic radiation (EMR) source 12 configured to emit a beam 13 of electromagnetic radiation or light, a mirror 14 spaced along an optical path that is capable of re-directing the beam 13, and a beam spreader 16 that is configured to increase the dimensions of the beam 13 so that the beam impinges a surface 17 of tissue 18 over a relatively large surface area. The beam exiting the beam spreader 16 or illuminating the surface 17 has a uniform or substantially uniform areal intensity profile. The intensity profile provides a two-dimensional map of the radiation intensity of the spread beam exiting the beam spreader 16 or across the surface area of the skin illuminated by the spread beam and may be expressed as an intensity density, i.e., intensity per unit area. Substantial uniformity is achieved by a variation in the intensity that is +30% or smaller of an average intensity of the spread beam.

The EMR source 12 may be a laser configured to emit the beam 13 of electromagnetic radiation by stimulated emission. The beam 13 dominantly propagates in a direction toward the mirror 14. The electromagnetic radiation in the beam 13 from the laser possesses a characteristic wavelength with a small optical bandwidth. Contingent upon the type of laser representing the EMR source 12, the electromagnetic radiation may lie within any of the infrared, visible, or ultraviolet bands of the electromagnetic spectrum. Most of the optical power in the beam 13 from the laser is concentrated over a small cross-sectional area and can be described by an optical intensity or electric field profile in a plane perpendicular (i.e., transverse) to the beam axis with a power described by a Gaussian function. The beam 13 from the laser exhibits a high degree of spatial coherence with extremely low beam divergence and a high degree of temporal coherence because of the small optical bandwidth. The beam 13 may have a Gaussian or non-Gaussian beam profile as understood by a person of ordinary skill in the art and may have any beam size (e.g., width, aspect ratio) recognized by a person of ordinary skill in the art. The beam spreading described herein is not constrained by properties of the beam 13 other than incident angle and wavelength.

Representative lasers appropriate to employ as the EMR source 12 include, but are not limited to, Nd:YAG lasers, semiconductor lasers, fiber lasers, Er:YAG lasers, Er:glass lasers, lamp-pumped lasers, diode pumped lasers, free electron lasers, optical fiber lasers, dye lasers, gas lasers such as argon and oxygen lasers, quantum cascade lasers, and quantum dot lasers. The EMR source 12 may also comprise a single high power multi-mode diode laser that is direct or fiber waveguide coupled with the beam spreader 16, a directed array of diode lasers (e.g., bars, VCSELs, stacks), or a A multimode fiber-coupled array of lasers. The laser(s) serving as the EMR source 12 may emit continuously (CW) or the emitted laser beam may consist of a fast sequence of pulses. Each type of laser serving as the EMR source 12 typically includes a gain medium capable of amplifying the power of light and compensate for resonator losses. The gain medium is electrically or optically pumped to inject energy into the amplified light circulating in the laser's optical resonator. Each type of laser serving as the EMR source 12 may be tunable in that the wavelength of operation can be altered or adjusted in a controlled manner over a range of possible wavelengths. The laser power may be in a range of, for example, 20 watts to 400 watts.

A controller 20 is connected with the EMR source 12 and communicates with the EMR source 12. The control over the EMR source 12 exerted by the controller 20 may provide the electrical or optical pumping, a tuning capability, and control over other parameters/components necessary for the operation of the EMR source 12 as understood by a person of ordinary skill in the art. Controller 20 includes a processor and memory for storing software and operational protocols for different tissue treatments using the EMR source 12. In one embodiment, the controller 20 is electrically connected for unidirectional or bidirectional communication with the EMR source 12 by any appropriate wired connection (e.g., universal serial bus communications, an IEEE 1394 interface, a networking standard like IEEE 802.3 Ethernet, serial signaling standards like RS-232 or RS-485, data acquisition input/output boards, etc.) that relies on electrical conductors, wires or cables extending from the controller 20 to the EMR source 12 to establish a communication path for data and control signals. In another embodiment, the controller 20 is electrically connected for unidirectional or bidirectional communication with the EMR source 12 by any appropriate wireless connection or communications protocol (e.g., IEEE 802.11 standard (WiFi), Bluetooth®, infrared, radio frequency, etc.) in which electromagnetic waves carry data and control signals over all or part of the communication path between the controller 20 and EMR source 12.

The EMR source 12, mirror 14, beam spreader 16, and optionally the controller 20 are located inside a housing 24 so that the path of the beam 13 of electromagnetic radiation is contained inside the housing 24 until propagation out of the housing 24 though the beam spreader 16. The light source 10 may be powered by an internal battery, an external power source, or a combination of both, and may include a switch to selectively power the light source 10 to operate for a period of time by operator action, by proximity of the light source 10 to the surface 17, or by direct contact of the light source 10 with the surface 17. The housing 24 of the light source 10 may be handheld so as to be compact and light enough to be operated to execute a tissue treatment while held by one or both of the operator's hands. Alternatively, the housing 24 may be adapted to be attached to the surface 17 with a temporary adhesive or otherwise fastened to the patient so as to reside with the beam spreader 16 in proximity to the surface 17.

The propagation direction of the electromagnetic radiation in the beam 13 is altered by the mirror 14 so that the beam 13 is incident on the beam spreader 16 with a direction of propagation 25. The mirror 14 may be constituted by any type of reflective surface capable of redirecting substantially the entire beam 13 at the beam's wavelength.

The beam spreader 16 includes an angular-selective element 32 defining an inlet in the representative form of an entrance surface 30 to the beam spreader 16. The beam 13 is transmitted through the angular-selective element 32 in the direction of propagation 25 with low intensity loss. In the representative embodiment, the beam 13 has a normal or near-normal angle of incidence relative to the entrance surface 30. The angular-selective element 32 has the form of an optical element that is used to transmit radiation at the wavelength of the EMR source 12 while blocking or attenuating radiation outside of a wavelength band.

In a representative embodiment, the angular-selective element 32 is constituted by a notch filter, which may be an optical coating containing a stack of layers comprised of dielectric materials characterized by different dielectric constants. In a specific embodiment, the notch filter representing the angular-selective element 32 may be comprised of a stack of dielectric layers formed by multiple layers of metal oxides such as Ta₂O₅ and SiO₂. The angular-selective element 32 is configured to transmit electromagnetic radiation over a narrow spectral transmission window of, e.g., 10 nm to 20 nm, with sharp spectral cut-on and cut-off regions and over a narrow incidence cone of entrance angles, e.g., an incidence cone of ±5° or smaller, or an incidence cone of ±10° or smaller. The incidence cone may also be referred to as angular acceptance. The selection of the thickness and composition of the dielectric layers in the stack can be used to tailor and tune the transmission and reflection characteristics of the angular-selective element 32. In any event, a substantial portion of the power in beam 13 is contained within an angular divergence that is equal to or less than the angular acceptance of the angular-selective element 32.

In the representative embodiment, the entrance surface 30 is planar or flat that is planar with a surface area bounded by an outer perimeter and the beam 13 impinges the entrance surface 30 with a normal angle of incidence or, alternatively, with a small non-normal angle of incidence. In other alternative embodiments, the entrance surface 30 may have either a concave curvature or a convex curvature. In this instance, the EMR source 12 is modified to emit the electromagnetic radiation in beam 13 with a curvature that matches the curvature of the entrance surface 30 to comply with the angular restriction on the incidence cone of the angular-selective element 32.

The beam spreader 16 further includes a scattering element 34 that is comprised of a scattering medium that efficiently scatters the radiation in the beam 13 during transmission through the thickness of the scattering element 34 and spreads the energy of beam 13 over a larger area. The angular-selective element 32 is spatially disposed between the scattering element 34 and the EMR source 12. The beam 13 is transmitted to the scattering element 34 through the angular-selective element 32. The probability of photon scattering in the scattering element 34 is approximately 100 percent such that substantially all of the photons contained in the beam 13 undergo at least one scattering event that changes their propagation direction within the beam 13. Preferably, the scattering events experienced by the beam 13 during propagation through the scattering element 34 occur with minimal absorption of the electromagnetic radiation by the scattering medium of the scattering element 34. Nevertheless, the optical power of the beam 13 is expected to be attenuated slightly during propagation of the beam 13 through the scattering medium of the scattering element 34.

In at least one embodiment, the scattering element 34 and angular-selective element 32 may be separate components and are positioned within the housing 24 such that the scattering element 34 and angular-selective element 32 have an adjoining relationship. In one specific embodiment, the angular-selective element 32 and scattering element 34 are bonded together to form an assembly in which the angular-selective element 32 and scattering element 34 contact across a two-dimensional interface 36. In the representative embodiment, the angular-selective element 32 and scattering element 34 are arranged such that the two-dimensional interface 36 is defined by a rectangular plane. As appreciated by a person having ordinary skill in the art, the angular-selective element 32 and scattering element 34 may be formed so that the two-dimensional interface 36 can assume any of a variety of geometrical shapes as appropriate for the particular treatment application.

The angular-selective element 32 operates to confine electromagnetic radiation backscattered within the scattering element 34 toward, and incident upon, the angular-selective element 32. Specifically, backscattered photons are reflected at the two-dimensional interface 36 between the angular-selective element 32 and the scattering element 34 back into the bulk of the scattering element 34. The angular-selective element 32 recycles the backscattered radiation based upon local angle of incidence relative to the two-dimensional interface 36. Similar to the beam 13 launched into the angular-selective element 32 from the EMR source 12, the reflection of backscattered photons at the interface 36 is incident angle dependent such that a small fraction of the backscattered radiation is transmitted through the angular-selective element 32 and lost to the surrounding environment. Typically, the reflectivity of the two-dimensional interface 36 will be on the order of 90 percent or higher.

The composition of the scattering medium of the scattering element 34 can be adjusted to provide material properties to match the wavelength(s) of the electromagnetic radiation in the beam 13 so that radiation is efficiently scattered at the wavelength(s) of the electromagnetic radiation in the beam 13. In representative embodiments, the scattering medium constituting the scattering element 34 may be alumina, quartz, polymethyl methacrylate (PMMA), or a glass such as frosted glass or milk glass. Frosted glass is produced by the sandblasting or acid etching of clear sheet glass. Milk glass is an opaque or translucent milky white or colored glass. The scattering element 34 and the angular-selective element 32 are comprised of materials with different scattering properties as the angular-selective element 32 has an angular sensitivity to the propagation direction of the photons or rays in the beam 13 that is absent in the scattering element 34. In addition, the angular-selective element 32 transmits the beam 13 within the incidence cone without significant scattering and/or attenuation and, in comparison to conventional beam spreaders, the scattering element 34 efficiently scatters the beam 13 received from the angular-selective element 32 to generate the spreading of the electromagnetic radiation in the beam 13.

The scattering element 34 includes an outlet in the representative form of an exit surface 38 that is planar and has a surface area bounded by an outer perimeter. In the representative embodiment, the exit surface 38 is rectangular with a width, w, and a length, l, to supply a surface area given by the product of the width, w, and length, l. Alternatively, the exit surface 38 may have other suitable two-dimensional geometrical shapes as understood by a person having ordinary skill in the art. In various embodiments, the surface area of the exit surface 38 may range from 1 cm² to 100 cm² and may be at least two orders of magnitude larger than the transverse extent of the beam 13 at the entrance surface 30. The transverse extent of the beam 13 at the entrance surface 30 may be given by a mode radius, which represents the radius at which the intensity drops to 1/e² of the intensity on the beam axis.

The beam 13 is effectively spread by the scattering element 34 such that a beam 13a containing electromagnetic radiation is output from the exit surface 38 and propagates toward the surface 17 of the tissue 18. The net propagation direction of the beam 13a is toward the surface 17. The intensity of the expanded beam 13 is approximately uniform across the surface area of the exit surface 38 and is projected from the exit surface 38 to illuminate the surface 17 as a light field over an approximately equivalent surface area. The scattering medium of the scattering element 34 scatters the electromagnetic radiation in the beam 13 to provide a two-dimensional spatial distribution for intensity in the beam 13a that is substantially uniformly across the exit surface 38. Beam 13a has a reduced directionality in comparison with beam 13.

The beam spreader 16 operates to convert the electromagnetic radiation in the beam 13 into the beam 13a of reduced directionality that is emitted with a substantially uniform distribution over the surface area of the exit surface 38 to allow uniform irradiation of a large volume/surface area of tissue 18. The beam spreader 16 converts beam 13 of high degree of spatial coherence into beam 13a that exhibits low spatial coherence and an increased divergence. However, the reduced directionality is achieved without a loss of temporal coherence.

In the representative embodiment, the exit surface 38 is separated by a short gap from the surface 17 of tissue 18. Alternatively, the exit surface 38 may have a contacting relationship with the surface 17 of tissue 18. The sidewalls of the beam spreader 16 are configured to laterally confine the beam 13 and limit lateral escape.

The radiation in the expanded beam 13a striking the surface 17 may be used to perform a treatment, such as a phototherapy treatment, in a manner understood to a person having ordinary skill in the art. The type of treatment may depend upon the wavelength and intensity of the delivered radiation. In one embodiment, the expanded beam 13a of electromagnetic radiation may be used for diathermy to produce heat in tissue 18 for therapeutic purposes to treat, for example, chronic arthritis, bursitis, fractures, gynecologic diseases, sinusitis, and other conditions. In other embodiments, the expanded beam 13a of electromagnetic radiation may be used for hair removal or other types of body or dermatological treatments, such as cellulite treatments, circumferential reduction, skin tightening fat reduction, and acne, psoriasis, acne, dermatitis, eczema, or vitiligo treatments. In another embodiment, the radiation in the expanded beam 13a is absorbed by the tissue to perform the treatment. The reduced directionality of beam 13a has a minimal impact on body or dermatological treatments that rely on penetration beneath the surface 17 because the skin is a highly scattering medium, and any light rays entering the skin at normal incidence are scattered close to the surface 17 and thus are redirected into all angles with increasing penetration from surface 17.

The treatment may be executed by an operator slowly moving the light source 10 across the surface 17 while continuously emitting the spread beam 13a of electromagnetic radiation in a painting motion. Alternatively, the light source 10 may be used to illuminate an area of the surface 17 with the beam 13a while being held substantially motionless and then moved to treat another area of the surface 17 in a seriatim stamping approach.

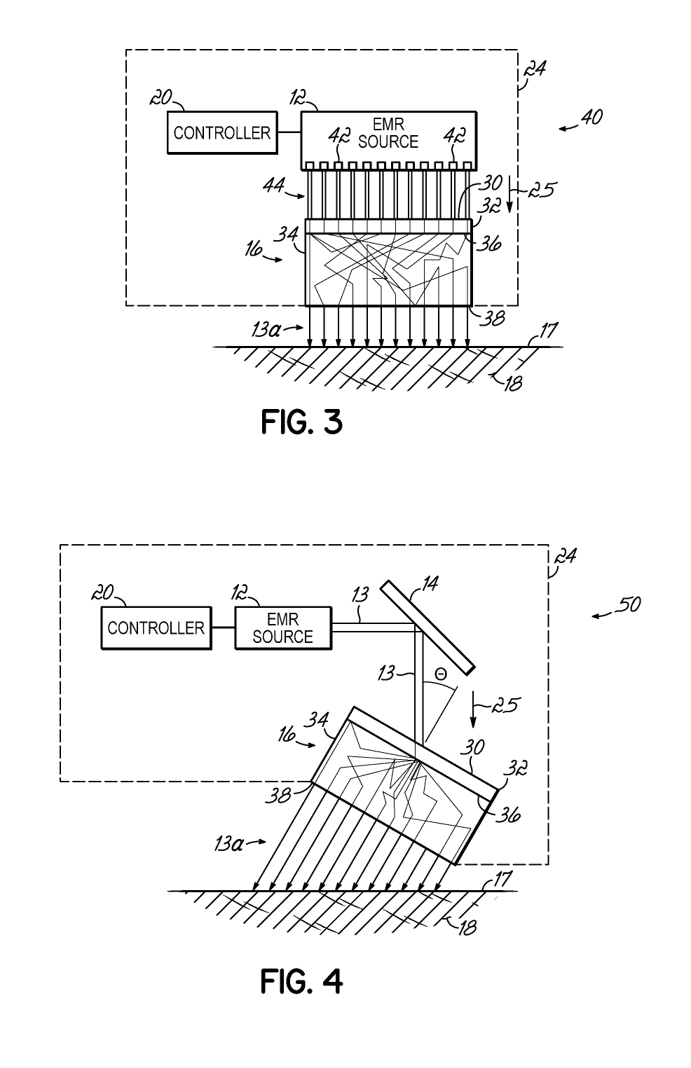

In an alternative embodiment and with reference to FIG. 2, the tissue 18 beneath the surface 17 may operate as a scattering medium that eliminated the need for the scattering element 34 of the beam spreader 16. The angular-selective element 32 is retained to confine electromagnetic radiation backscattered within the tissue 18 and out of the surface 17 toward the angular-selective element 32. The angular-selective element 32 may be separated from the surface 17 by a gap, as shown in FIG. 2. The backscattered electromagnetic radiation is redirected by the angular-selective element 32 toward the surface 17. The principles of this alternative embodiment may be applied to light sources 40 (FIG. 3) and 50 (FIG. 4).

With reference to FIG. 3 in which like reference numerals refer to like features in FIG. 1 and in accordance with an alternative embodiment of the invention, a light source 40 includes a plurality of EMR sources 42 that are configured to emit a plurality of beams 44 of electromagnetic radiation at spatially distributed locations. In one embodiment, the EMR sources 42 are constituted by a plurality of semiconductor lasers mounted on a single circuit board. The multiple light beams 44 are injected from the EMR sources 42 into the beam spreader 16 and communicated or transported in the direction of propagation 25 through the angular-selective element 32 into the scattering element 34. The coherent light in each of the beams 44 is transformed by the cooperation of the angular-selective element 32 and scattering element 34, as described above, into the beam 13a of coherent light that is emitted over the surface area of the exit surface 38 of beam spreader 16.

In the representative embodiment, the EMR sources 42 are arranged in a linear array. However, the invention is not so limited as the EMR sources 42 may be arranged, for example, in a ring-shaped array. In the representative embodiment, the EMR sources 42 are configured to operate at the same nominal wavelength. However, the invention is not so limited as the EMR sources 42 may operate at different wavelengths.

With reference to FIG. 4 in which like reference numerals refer to like features in FIG. 1 and in accordance with an alternative embodiment of the invention, a light source 50 includes the EMR source 12, mirror 14, beam spreader 16, and controller 20. The angle of incidence, θ, of the light beam 13 relative to the entrance surface 30 is increased to an angular value greater than 10° and, in certain embodiments, an angular value in the range of 20° to 30°. In the representative embodiment, the beam spreader 16 is tilted relative to the direction of propagation 25 of the beam 13. The design of the angular-selective element 32 is adjusted such that the coherent light in the beam 13 is transmitted or communicated through the angular-selective element 32 to the scattering element 34. The greater angle of incidence, θ, ensures that the backreflected radiation is not fed back into the EMR source 12.

References herein to terms such as “vertical”, “horizontal”, etc. are made by way of example, and not by way of limitation, to establish an absolute frame of reference. It is understood by persons of ordinary skill in the art that various other frames of reference may be equivalently employed for purposes of describing the embodiments of the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “includes”, “having”, “has”, “with”, “comprised of”, or variants thereof are used in either the detailed description or the claims, such terms are intended to be inclusive in a manner similar to the term “comprising.”

While the invention has been illustrated by a description of various embodiments and while these embodiments have been described in considerable detail, it is not the intention of the applicant to restrict or in any way limit the scope of the appended claims to such detail. Additional advantages and modifications will readily appear to those skilled in the art. Thus, the invention in its broader aspects is therefore not limited to the specific details, representative apparatus and method, and illustrative example shown and described. Accordingly, departures may be made from such details without departing from the spirit or scope of applicant's general inventive concept.

What is claimed is:

1. A method for spreading at least one beam of electromagnetic radiation, the method comprising:
directing the electromagnetic radiation of a first beam to impinge an inlet to the angular-selective element at an angle of incidence greater than 10°;
transmitting the electromagnetic radiation in a propagation direction through the angular-selective element and into a scattering element;
scattering the electromagnetic radiation in a scattering medium of the scattering element such that an area of a two-dimensional spatial distribution of intensity output at an outlet of the scattering element is substantially uniformly across the outlet; and
reflecting a majority of the electromagnetic radiation that is scattered by the scattering medium from the angular-selective element in a direction opposite to the propagation direction.

2. The method of claim 1 wherein the angle of incidence is in a range of 20° to 30°.

3. The method of claim 1 wherein the angular-selective element is tilted relative to the propagation direction.

4. The method of claim 1 wherein the angular-selective element and the scattering element are tilted relative to the propagation direction.

5. The method of claim 1 wherein the angular-selective element is configured to transmit the electromagnetic radiation to the scattering element over a spectral transmission window of 10 nm to 20 nm and over an incidence cone of entrance angles of ±10° or smaller.

6. The method of claim 1 further comprising:
tilting the angular-selective element relative to the propagation direction.

7. The method of claim 1 further comprising:
outputting the electromagnetic radiation from the outlet toward a skin surface to perform a dermatological treatment.

8. The method of claim 1 wherein the angular-selective element is a notch filter, and further comprising:
generating the electromagnetic radiation of the first beam with a laser.

9. The method of claim 1 wherein the scattering element is comprised of alumina, quartz, polymethyl methacrylate (PMMA), or glass.

10. The method of claim 1 wherein transmitting the electromagnetic radiation of the beam in the propagation direction through the angular-selective element and into the scattering element comprises:
transferring the electromagnetic radiation of the first beam across a two-dimensional interface along which the scattering element adjoins the angular-selective element, and the majority of the electromagnetic radiation backscattered by the scattering element is reflected at the two-dimensional interface.

11. The method of claim 1 wherein transmitting the electromagnetic radiation of the beam in the propagation direction through the angular-selective element and into the scattering element comprises:
transferring the electromagnetic radiation of the first beam across a two-dimensional interface along which the scattering element contacts the angular-selective element, and the majority of the electromagnetic radiation backscattered by the scattering element is reflected at the two-dimensional interface.
